(19) United States
(12) Patent Application Publication
(10) Pub. No.: US 2023/0371805 A1
(43) Pub. Date: Nov. 23, 2023

(54) INTUBATION DEVICE AND SYSTEM

(71) Applicant: Flexicare (Group) Limited, Mountain Ash, Mid Glamorgan (GB)

(72) Inventors: Ghassem Poormand, Mountain Ash (GB); Hash Poormand, Mount Ash (GB); Tom Edmunds, Mountain Ash (GB); Geraint Whitcombe, Mountain Ash (GB); Julian Long, Mountain Ash (GB)

(21) Appl. No.: 18/027,567

(22) PCT Filed: Sep. 23, 2020
(86) PCT No.: PCT/EP2020/076585

§ 371 Date: Mar. 21, 2023

(51) Int. Cl. A61B 1/267; A61B 1/00; A61B 1/05

(52) U.S. Cl. CPC A61B 1/267; A61B 1/00052; A61B 1/00032; A61B 1/05; A61B 1/06

(57) ABSTRACT

The present invention relates to: a laryngoscopy system including a laryngoscope body and an image display module; an image display module for a laryngoscopy system; and a kit including an image display module and at least one laryngoscope body. The laryngoscope body has a handle portion and a blade portion, the blade portion having a distal end and a handle connection end and a blade body extending therebetween. The laryngoscope body further comprises an imaging component disposed on or housed by the blade portion. The image display module is configured to receive image data from the imaging component, and includes an accelerometer for motion input control of the image display module.

### FIELD OF THE INVENTION

The present invention relates to intubation devices, and in particular relates to laryngoscopes, blades for laryngoscopes, and a laryngoscopy system.

### BACKGROUND

Laryngoscopes are widely used to assist with tracheal intubation. A conventional laryngoscope (also commonly referred to as a ‘direct vision’ laryngoscope) consists of a handle and a blade connected at a one end of the handle. In typical use, a clinician standing above/behind the head of a supine patient holds the laryngoscope in one hand, blade-downwards with the blade underside away from them, and an endotracheal tube (ET tube) in the other hand. The blade is then inserted into the patient's airway, and may be turned and/or lifted to move tissues such as the tongue and epiglottis out of the way, to provide a direct line of sight along the patient's airway to the patient's vocal cords to enable the clinician to visualise the ET tube as it is passed through the vocal cords into the trachea. It is important to ensure that the ET tube is correctly located in the patient's trachea, and so visualisation of this process is important. Typically, the laryngoscope blade carries a light, to assist visibility. Care is needed during this procedure not to damage the patient's teeth by using them as a reaction point for the rear end of the blade when lifting the tongue.

The process as described above where the ET tube placement is visualised directly is termed ‘direct vision laryngoscopy’. However, direct vision laryngoscopy is not always possible, depending on the particular anatomy of the patient. For example, difficulties may be encountered where the patient has restricted neck flexibility, or where the patient is obese. In such cases, indirect vision laryngoscopy may be employed, i.e. the patient's vocal cords are visualised other than by a direct line of sight. Video laryngoscopes are well-known devices for use in indirect-vision laryngoscopy. A typical video laryngoscope is similar to a conventional direct-vision laryngoscope, but further comprises a camera or imaging sensor located on or near the laryngoscope blade, arranged to transmit images to an external screen or monitor for observation by the clinician performing the procedure.

Intubation is almost always urgent. With the urgent situation and substantial force applied, there is a possibility of minor abrasions in the mouth or throat. For this reason, sterility is particularly important, otherwise a laryngoscope may contribute to carrying infection from the patient to another patient. Due to this need for sterility, many direct-vision laryngoscopes are now “single use”: sometimes just the blade and sometimes the entire device. Reusable components pose a higher contamination risk than single use components, because it can be difficult to decontaminate such components to a satisfactory extent between successive uses of the equipment on different patients.

However, provision of a sterile (or substantially sterile) system poses a particular problem for video-based intubation devices (e.g. laryngoscopes having a camera or imaging sensor arranged to transmit images to an external screen or monitor for observation), because many components of such are typically not designed for single use due to the cost involved.

One solution to this problem has been to provide a reusable handle with an attached central blade shaft containing a camera or imaging sensor, over which a disposable, single-use blade-carrying sheath is located. This allows for at least a portion of the device to be single use, and the risk of cross-patient contamination to be reduced accordingly. Such blade sheathes are typically made from transparent moulded plastic, in particular because they must allow image transmission through the blade-carrying sheath. However, there are a number of problems with such a design; in particular, the central blade shaft offers a particular contamination risk, as during use it is located with a patient's airway. Furthermore, there is a small risk of dislocation of the blade-carrying sheath from the central blade shaft during use, which is a potential safety risk.

Another problem with some known video laryngoscopy systems is that such systems typically comprise one or more external buttons or operational control members (for example, power buttons or switches), used to operate the system (for example, to turn the system on or off). However, the presence of external buttons or other operational control members such as switches or dials may, in some cases, provide a contamination risk. It can be difficult to ensure adequate cleaning or sterilisation has occurred in the small groove or gap typically surrounding the button or operational control member.

The present invention has been devised in light of the above considerations.

### SUMMARY OF THE INVENTION

An aim herein is to provide new and useful intubation devices including laryngoscopes, blades for laryngoscopes, and laryngoscopy systems, which address some or all of the issues discussed above.

The proposals set out herein relate in general to laryngoscopes of the following type, including a laryngoscope body comprising a handle portion and a blade portion.

The blade has a front or tip end (distal end) and a rear end (handle connection end), and a blade body extending between them. The blade body has an underside and an upper side. In this disclosure, the terms ‘upper side’ and ‘underside’ are used in relation to the blade as it would typically be oriented in use: with the handle vertical and blade body affixed to the downwards end of the handle, the blade projecting in the operating position.

The upper side of the blade typically comprises a generally flat or arcuate surface, which, in use, engages with the patient's airway anatomy. This surface may be substantially flat in a transverse direction. The shape of the underside of the blade may vary according to the precise design of the laryngoscope in question. However, the underside may have one or more housing portions for e.g. light emitting components of the laryngoscope. Preferably, the underside of the blade has a laterally-directed guide face for guiding intubation. Such guide face may be provided by a housing portion which houses e.g. light emitting components of the laryngoscope such as LEDs, or imaging components such as a camera or imaging sensor.

The blade may be of any one of a number of suitable materials, however preferably, the blade is of metal or metal alloy. For example, the blade may be made from zinc. The blade may be formed by press-moulding from sheet in a manner that is known, but preferably is formed by casting or moulding. Casting or moulding the blade can give greater freedom of shape design.

Preferably the blade has a low vertical profile. In other words, the total vertical extent of the blade at any one point measured along the longitudinal length of the blade between the underside and the upper side of the blade may be 20 mm or less, 15 mm or less, or 10 mm or less. Reducing the vertical profile of the blade allows for greater freedom of movement during the intubation process given the limited space within which the clinician can work.

The blade and handle are connected at the rear end of the blade. This connection may by a fixed connection, or may be a pivotable connection. Where the connection is a pivotable connection, the blade may be pivotable relative to the handle between an operating position and a folded position. The size and shape of the handle portion of the laryngoscope is not particularly limited. The handle may have a conventional form, i.e. may be generally cylindrical or barrel-shaped.

Whilst the proposals described herein relate in general to the kind of laryngoscope described above, some proposals may be more generally applicable. Additionally, the proposals described herein should be considered as a series of independently combinable proposals containing a number of options and preferences. It is considered than each of these proposals, including the options and preferences discussed therein, may be applied alone or in combination.

Accordingly, in a first aspect, the present invention provides a laryngoscopy system comprising:
a laryngoscope body having a handle portion and a blade portion, the blade portion having a distal end and a handle connection end and a blade body extending therebetween, the laryngoscope body further comprising an imaging component disposed on or housed by the blade portion; and
an image display module configured to receive image data from the imaging component;
wherein the image display module includes an accelerometer for motion input control of the image display module.

In a second aspect, the present invention provides an image display module configured for engagement with a laryngoscope body comprising an imaging component to thereby form a laryngoscopy system;
wherein the image display module is configured to receive image data from the imaging component of the laryngoscope body when engaged with the laryngoscope body; and
wherein the image display module comprises an accelerometer for motion input control of the image display module screen.

An image display module is a module suitable for displaying images transmitted electronically or otherwise (e.g. by wired connection) from the imaging component of the laryngoscope body. The image display module has a screen portion on which the images can be displayed when the image display module is in use.

A laryngoscopy system in which the image display module includes an accelerometer for motion input control of the image display module offers a number of advantages over known systems. It may offer increased ease of use of the laryngoscopy system as compared with laryngoscopy systems having conventional controls. The term ‘motion input control’ is used herein to define that one or more operational features of the image display module are controllable based on motion detected by the accelerometer. The operational features which may be controllable in such manner are not particularly limited. In some cases, the operational status of the image display module may be controllable based on motion activity detected by the accelerometer. For example, the system may be configured such that when the accelerometer detects motion corresponding to being picked up, the power status of the system is set to ‘on’. When the accelerometer detects that no motion has occurred for a predetermined time period, the power status of the system may be set to ‘off’. Such operation may be achieved in a conventional manner e.g. by appropriate configuration of an internal processor of the image display module.

In one preferred arrangement, the system comprises an active mode and a sleep mode (or ‘standby’ mode). The active mode may be triggered by detected motion activity of the image display module. Upon entering active mode, or when further motion activity is detected when the system is already in the active mode, the system may set a movement level threshold, and update a system timer status. A movement threshold level is a minimum amount of movement required to trigger entry of the system into the active mode, or maintain the system in active mode. The system timer status may be set to a predetermined time value, which may be any predetermined time value of 1 second or more. For example, the predetermined time value may be 10 seconds, 20 seconds, 30 seconds, 60 seconds, 120 seconds, 180 seconds, 240 seconds, 300 seconds etc. The system may be configured such that if no further motion activity of the image display module is detected within the predetermined time, the system may enter a ‘sleep’ or ‘standby’ mode, in which a display of the image display module is turned off, and in which the processor of the image display module is in a sleep mode. In some arrangements, the system may perform a ‘fade out’ procedure in which the display of the image display module fades to black, prior to entering the sleep or standby mode. The longer the predetermined time value, the greater battery drain on the system during non-use (due to the greater length of time that that a display of the image display module is on, and in which the processor of the image display module is active, during non-use). The shorter the predetermined time, the greater risk that the system will enter a sleep/standby mode during a routine pause in procedure, thereby disrupting user experience. It has been determined that a particularly preferred time value is about 60 seconds—i.e. if the accelerometer is not triggered by motion activity above a movement level threshold within 60 seconds, the image display module will enter a ‘sleep’ or ‘standby’ mode (optionally after performing a ‘fade-out’ procedure as mentioned above). It has been determined that 60 seconds provides a reasonable balance between optimisation of battery life and minimising disruption to user experience during use of the system.

The system may further comprise a ‘hard off’ mode, in which the image display module is not controllable by motion input control via the accelerometer. The system may be operable to be placed in such a ‘hard off’ mode during e.g. shipping or storage of the system. This can assist in avoiding unintentional triggering of an active mode of the system due to movement during shipping or storage. The system may be placed in the ‘hard off’ mode upon manufacture. The system may be configured to be moved between a ‘hard off’ mode and a standard mode of operation by e.g. connection to a charging apparatus. For example, the system may be configured such that the first time it is placed on charge, standard functionality of the system (including in some cases operation of active modes and sleep modes as set out above) is triggered.

The image display module may be a control module of the laryngoscopy system. The image display module may be operable to control one or more functional features of the laryngoscope body, including but not limited to control of operation of, or control of power supply to, components of the laryngoscope body including light emitting components such as LEDs, and/or imaging components such as a camera or imaging sensor. Control of operation of imaging components may include control of image data transmission and/or storage e.g. control of the start and/or end of a recording session in which image data is transmitted to and stored by the image display module. For example, the image display module may be configured to start and/or end a recording session based on a user input to the image display module e.g. via motion input or touch input to a touch-sensitive portion of the image display module. Control of light emitting components may include e.g. control of brightness of one or more light emitting components of the system. For example, brightness of one or more light emitting components of the system may be controlled based on a user input to the image display module e.g. via motion input or touch input to a touch-sensitive portion of the image display module.

The image display module may have a touch-sensitive portion, e.g. a touch-sensitive screen portion, which allows touch-input control of the laryngoscope. In particular, the image display module may be controllable by touch input. Where the image display module is configured to be able to record and/or store images or video, such touch-screen input may be used to control recording function of the device.

The image display module may have internal memory and/or storage for storing data e.g. video files. The image display module may be configured to be able to record and store images and/or videos from the imaging component. The image display module may be configured to allow transfer of stored data to one or more external storage devices, e.g. via USB or any other suitable connection.

Laryngoscopy systems according to the present invention also offer advantages in respect of providing reduced risk of contamination, because providing motion input control of the image display module may reduce or eliminate the need for the presence of control components such as external buttons, switches or dials on the image display module, or elsewhere in the laryngoscopy system, for example on the laryngoscope body. As discussed above, the presence of such members may provide a contamination risk, as it is often difficult to ensure adequate cleaning or sterilisation has occurred in the small groove or gap typically surrounding the button or operational control member.

A laryngoscopy system according to the present invention may have no external buttons, switches or dials. The term ‘external’ is here used to define a feature which is disposed on or forms part of an outer surface of the laryngoscopy system. A laryngoscopy system according to the present invention may comprise one or more internal buttons or switches. For example, it may comprise an internal (recessed) device reset button, accessible via a pin-hole opening formed in the outer surface of the image display module. Conventional laryngoscopy systems commonly have one or more external buttons, switches or dials formed or located on an outer surface of the laryngoscopy system, for example a power button for controlling the power status of the system, or a recording button for control of image data transmission and/or storage. In conventional laryngoscopy systems, the presence of such control components (buttons, switches or dials) is generally necessary to allow proper function of the system. In contrast, a laryngoscopy system according to the present invention requires fewer or no such control components, because the system is controllable by motion input control. By providing a laryngoscopy system having fewer or no external buttons, switches or dials, the contamination risk of the system can be reduced.

Whilst for certain applications it may be preferable for the laryngoscopy system to have no external control members as discussed above, it is contemplated that in some cases, the laryngoscopy system may comprise one or more external control members. For example, the laryngoscopy system may comprise a single on/off button configured for control of a power status of the image display module. Such arrangements may be beneficial where the laryngoscopy system is intended for use as a high-portability system (for example, for use by emergency services personnel), as providing user-activated control of the power status of the image display module, rather than relying solely on accelerometer-based control, can help to reduce battery drain on the system by allowing manual powering down of the system immediately after use.

The laryngoscopy system may include at least one power source for powering the system (e.g. for providing power to one or more of the image display module and/or the imaging component. Alternatively, the laryngoscopy system may be connectable to an external power source for powering the system. In some arrangements, the image display module comprises an integral power source. For example, the image display module may comprise an integral, rechargeable battery, such as a lithium ion battery, or any other suitable battery. In some arrangements, the power source may be removable. In other arrangements, the integral power source may be non-removable. Where the integral power source is non-removable, it may be charged by connection of the image display module to an external power source, such as a charging unit. Systems having a removable battery component which is removed between uses for charging of the battery can provide increased contamination risk, due to the presence of the battery recess. Therefore, provision of a non-removable integral power source may be preferable, as this can allow the image display module to be provided as a substantially sealed module.

Where the image display module comprises an integral power source, this may be the sole power source for the laryngoscopy system. In other words, there may not be a power source forming part of the laryngoscope body. This is in contrast to conventional laryngoscope arrangements, where it is typical for there to be a battery located in e.g. a handle portion of the conventional laryngoscope, to power imaging or light emitting components of the conventional laryngoscope. Removing the need for a power source located within the laryngoscope body is advantageous because it reduces the weight of the laryngoscope body. Furthermore, the component count of the laryngoscope body can be reduced. This can also lead to reductions in the cost of manufacture of the laryngoscope body, which makes it suitable for use as a ‘single use’, or disposable component. Furthermore, more design freedom is available for e.g. the shape of the laryngoscope handle, if it does not need to be designed to hold an internal power source.

The image display module may have an ingress protection rating as defined in international standard IEC 60529 of IP64 or higher. The IP Code consists of the letters IP followed by two digits and an optional letter. As defined in international standard IEC 60529, it classifies the degrees of protection provided against the intrusion of solid objects (including body parts like hands and fingers), dust, accidental contact, and water in electrical enclosures. A device having an IP64 rating is protected from total dust ingress, and protected from low pressure water jets from any direction.

The image display module may be pivotably mounted on the handle portion of the laryngoscope body. Alternatively, the image display module may be rigidly mounted on the handle portion of the laryngoscope body. A pivotable mounting may provide advantages in terms of ease of use of the system, as a user of the device can adjust the angle of the image display module relative to the laryngoscope body to thereby select a comfortable viewing angle of the image display module during use. Where the mounting is a pivotable mounting, it may be preferable for the pivot point or hinge to be located on the handle portion of the laryngoscope body, rather than on the image display module. This is because where the pivot hinge is located on the image display module, this can provide an additional contamination point on the image display module. The pivot point or hinge may be recessed into the handle portion of the laryngoscope body, to retain a low profile for the laryngoscope handle.

The image display module may removably mountable on the handle portion of the laryngoscope body. In one arrangement, the image display module may be mounted to the handle by engagement with corresponding male and female electrical connectors located on the image display module and on the handle portion of the laryngoscope body. For example, the image display module and laryngoscope handle may have corresponding male and female USB connectors, which are engageable to provide both an electrical and mechanical connection between the image display module and the laryngoscope handle.

The image display module may be configured to engage with the handle portion of the laryngoscope body such that a median plane of a screen portion of the image display is offset from a median plane of the handle portion of the laryngoscope body. The image display module may be configured to engage with the handle portion of the laryngoscope body such that a median plane of a screen portion of the image display may intersect with a longitudinal central portion of the blade body of the laryngoscope. A longitudinal central portion of the blade body is herein defined as a portion extending 25% of the maximum lateral extent of the blade body in each direction from a longitudinal centreline of the blade body. In some arrangements, a median plane of a screen portion of the image display may intersect the longitudinal centreline of the blade body. By providing a laryngoscopy system where the image display module is arranged in this manner, a clinician using the laryngoscopy system may be more easily able to manipulate the laryngoscope in response to the observed video being displayed on the image display module. Furthermore, the laryngoscope may be more intuitive to use than a known system where the image display module is greatly offset from the laryngoscope blade, or where an image from the imaging component is displayed on a separate, external screen.

The laryngoscope body may comprise one or more light emitting components disposed on or housed by the blade portion. The one or more light emitting component may be comprise e.g. light emitting diodes (LED), fibre optic emitters, or any other suitable light emitters. The one or more light emitting component may be ‘off-the-shelf’ or prefabricated light emitting components. Providing light emitting component(s) disposed on or housed by the blade portion can prevent the need for use of external light sources during use of the laryngoscopy system. Said one or more light emitting components may be powered by or via the image display module of the laryngoscopy system, e.g. by an internal power source of the image display module, or by an external power source connected to the image display module. In this way, it is not necessary for the laryngoscopy body to contain any power source.

The one or more light emitting components may be located at substantially the same longitudinal position along the blade as the imaging component. Alternatively or additionally, one or more light emitting components may be located at different longitudinal positions along the blade from the imaging component.

The precise form of the imaging component is not particularly limited. The imaging component may be an ‘off-the-shelf’ or prefabricated imaging component. The imaging component may comprise a charge-coupled device (CCD), or an active-pixel sensor (CMOS sensor). The imaging component may have a viewing angle of at least 50°, at least 60°, at least 70°, or at least 80°. The imaging component may be configured for RGB imaging. The imaging component may have a sensor area of at least 200×200 pixels, at least 300×300 pixels, or at least 400×400 pixels. In one embodiment, the imaging component comprises a 640×480 pixel CMOS sensor. Said imaging component may be powered by or via the image display module of the laryngoscopy system, e.g. by an internal power source of the image display module, or by an external power source connected to the image display module. In this way, it is not necessary for the laryngoscopy body to contain any power source.

The imaging component may be configured so that the field of view of the imaging component includes part of the blade portion of the laryngoscope body. For example, the field of view of the imaging component may include at least part of the distal end of the blade. In this way, a user of the laryngoscopy system can use the visible portion of the laryngoscope blade as a frame of reference when performing a laryngoscopy procedure.

In some arrangements, the imaging component may be configured so that a viewing axis of the imaging component (the central axis of the field of view of the imaging component) intersects with a longitudinal upright median plane of the blade, at a point at or near the distal end of the blade. A longitudinal upright median plane is here defined as a longitudinal plane passing through the mid-point of the blade body, and extending in a vertical direction. The viewing axis may intersect with the median plane a vertical distance of between 1 mm and 20 mm below the underside of the blade.

The imaging component may be contained within a housing section of the blade portion of the laryngoscope body. Housing the imaging component in a housing section can provide protection against damage to the imaging component. The imaging component may be fixed within the housing in a manner which prevents accidental misorientation of the imaging component. For example, the imaging component may be glued in place. Alternatively or additionally the imaging component may be held by one or more projections within a housing portion. For example, the imaging component may be held in a snap-fit arrangement within a housing section of the blade portion of the laryngoscope body. Such a snap-fit arrangement can improve ease of assembly of the laryngoscope.

Further proposals herein relate to the shape of the blade body, and are applicable both to the laryngoscopy system set out in relation to the first aspect of the invention, but are also generally applicable to ‘conventional’ laryngoscopes (i.e. direct vision laryngoscopes), or in general to any laryngoscopy system comprising a laryngoscope having a handle and a blade connected at a one end of the handle.

Accordingly, in a third aspect, the present invention provides a laryngoscope body having a handle portion and a blade portion, the blade portion having a distal end and a handle connection end and a blade body extending therebetween, and wherein the blade body comprises a locally narrowed portion disposed adjacent the handle connection end of the blade, the lateral extent of the locally narrowed portion being less than 60% of the maximum lateral extent of the blade body.

In some cases, the blade portion may be provided as a component separately from other components of a laryngoscopy system (for example, as a replacement blade). Accordingly, this aspect also includes a blade suitable for use in a laryngoscopy system, the blade having a distal end and a handle connection end suitable for connection to a handle portion of a laryngoscope body, and a blade body extending therebetween, wherein the blade body comprises a locally narrowed portion disposed adjacent the handle connection end of the blade, the lateral extent of the locally narrowed portion being less than 60% of the maximum lateral extent of the blade body.

The phrase ‘adjacent the handle connection end of the blade’ is used herein to define that the locally narrowed portion is disposed closer to the handle connection end of the blade than to the distal end of the blade. In other words, the locally narrowed portion may be in the rear 50% of the blade, as defined relative to the handle connection end. The lateral extent of the blade body is the width of the blade in a direction perpendicular to the longitudinal direction in which the blade extends. As described above, the blade body has an underside and an upper side, and the upper side typically comprises a generally flat or arcuate surface which, in use, engages with the patient's airway anatomy. For a Macintosh-style blade (‘Mac’ blade), conveniently the maximum lateral width of this surface will typically be substantially equal to the maximum lateral width of the blade.

A laryngoscope blade having a locally narrowed portion may provide improved ease of use during laryngoscopy procedures, by allowing a greater range of movement of the blade when positioned within the airway of a user. For conventional laryngoscope blades, the range of movement of the blade may otherwise be restricted by e.g. a patients teeth during a laryngoscopy procedure. This restriction in the range of movement can be reduced or avoided by provision of a locally narrowed portion of the blade because the blade may be rotated more easily without impinging on the patient's teeth. Furthermore, the risk of damage to a patient's teeth during use of the laryngoscope may be reduced.

The lateral extent of the locally narrowed portion may be 50% or less, 40% or less or 30% or less of the maximum lateral extent of the blade. Preferably the lateral extent of the locally narrowed portion is 10% or more, or 20% or more of the maximum lateral extent of the blade. If the width of the narrowed portion of the blade is too small compared to the rest of the blade, this can lead to increased risk of the blade breaking during use. The blade body may be asymmetrically narrowed, i.e. the locally narrowed portion may be formed asymmetrically with respect to the longitudinal axis of the blade body such that the blade body is not axisymmetric in a longitudinal direction. Alternatively, the blade body may be symmetrically narrowed.

The length of the locally narrowed portion may be in a range from about 10 mm to about 50 mm in length. In some arrangements, the locally narrowed portion may be about 20 mm, about 30 mm, or about 40 mm in length.

In a fourth aspect, which, similarly to the third aspect, is applicable both to the laryngoscopy system set out in relation to the first aspect of the invention, but also generally applicable to any laryngoscopy system comprising a laryngoscope having a handle and a blade connected at a one end of the handle, the present invention provides a laryngoscope body having a handle portion and a blade portion, the blade portion having a distal end and a handle connection end and a blade body extending therebetween, wherein the upper side of the blade body defines a generally longitudinally arcuate surface, and at least a portion of the surface subtending an angle of at least 30° has a constant radius of curvature.

The portion having a constant radius of curvature may subtend an angle of at least 35°, at least 40°, at least 45° or at least 50°. Providing a blade with such a curvature can allow for ease of insertion of the laryngoscope blade to a patients airway during e.g. an intubation procedure.

The portion of the blade having constant radius of curvature may be disposed towards the distal end of the blade. In some arrangements, the portion of the blade having constant radius of curvature may include the distal end of the blade. The portion of the blade having constant radius of curvature may include a central portion of the blade. The radius of curvature of the constant radius of curvature portion of the blade may be selected as appropriate for the intended use of the blade.

The blade body may have a ‘Macintosh’ (‘Mac’) blade profile. For example, the blade may have a Mac 3 profile, or a Mac 4 profile. A Mac 3 blade has a length of 138 mm+/−1 mm, with a constant radius portion of 101 mm+/−2 mm. A Mac 4 blade has a length of 154.5 mm+/−1 mm, with a constant radius portion of 101 mm+/−2 mm.

The longitudinally arcuate surface of the blade body may be substantially flat in a transverse direction. Alternatively, the longitudinally arcuate surface of the blade body may be curved in a transverse direction. Further proposals herein relate to a kit comprising an image display module according to the second aspect of the invention, and further comprising at least one laryngoscope body, the laryngoscope body having a handle portion and a blade portion, the blade portion having a distal end and a handle connection end and a blade body extending therebetween, the laryngoscope body further comprising an imaging component and a light emitting component disposed on or housed by the blade portion.

The kit may be provided with a plurality of laryngoscope bodies as defined above. In one arrangement, the laryngoscope body or bodies are provided in respective sealed and sterile packaging, e.g. in a container such as a sealed bag, or sealed box. In this way, a plurality of sterile laryngoscope bodies can be used as single-use laryngoscope bodies, in combination with a single reusable and sterilisable image display module.

The kit may further include other components for use in combination with one or both of the image display module and the laryngoscope body. For example the kit may comprise charging apparatus, configured to engage with the image display module to charge an internal battery of the image display module. Said charging apparatus may include one or more of a charging stand, a USB cable and/or a mains power supply connector or connection cable.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows a rear view line drawing of a laryngoscopy system according to the present invention, with the image display module shown in a detached configuration.

FIG. 2 shows a front view line drawing of the laryngoscopy system of FIG. 1, with the image display module shown in an attached configuration.

FIG. 3 shows a partial cutaway line drawing of the laryngoscopy system of FIGS. 1 and 2, showing an internal wiring configuration of the laryngoscope body.

FIG. 4 shows a side view line drawing of a laryngoscopy system according to the present invention, demonstrating range of movement of the image display module relative to the laryngoscope body.

FIG. 5 shows a side view line drawing of a laryngoscopy system according to the present invention, demonstrating constant radius of curvature of a portion of the blade body.

FIG. 6 shows a bottom view line drawing of a blade body having a locally narrowed portion.

FIG. 7 shows a schematic drawing of a kit according to the present invention.

FIG. 8 shows a schematic drawing of a laryngoscopy system according to the present invention in use.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

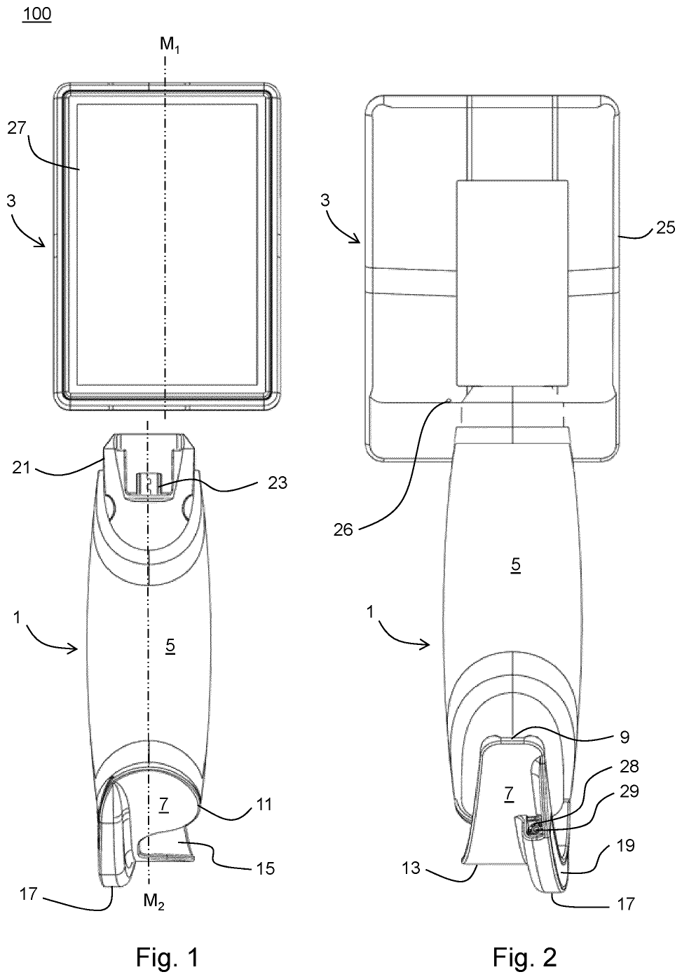

FIG. 1 and FIG. 2 show front and rear views of a laryngoscopy system 100 according to the present invention. The laryngoscopy system 100 includes a laryngoscope body 1 and an image display module 3, each of which will be described in detail below. The laryngoscope body 3 and the image display module are interengageable to form a single unit. In FIG. 1, the image display module is shown in a detached configuration. In FIG. 2, the image display module is shown in an attached configuration.

The laryngoscope body 3 comprises a handle portion 5 and a blade portion 7, the blade portion having a distal end 9 (also referred to herein as a front, or tip end) and a handle connection end 11 (also referred to herein as a rear end) and a blade body extending therebetween. The blade body has an underside 13 and an upper side 15, these being used in relation to the blade as oriented in use: with the handle vertical and blade body affixed to the downwards end of the handle, the blade projecting in the operating position.

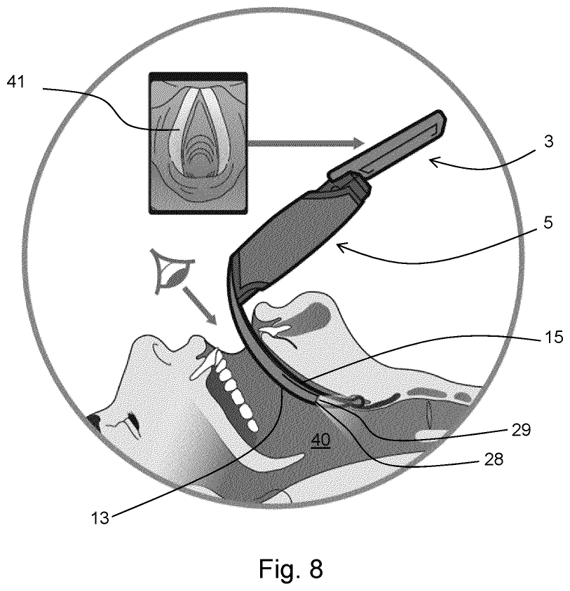

The upper side 15 of the blade portion 7 comprises a generally arcuate surface, which, in use, engages with the patient's airway anatomy, as shown in FIG. 8 (discussed below). This arcuate surface is substantially flat in a transverse direction. The underside 13 of the blade comprises a housing portion 17 which houses one or more light emitting components 28 (here, LEDs) and one or more imaging components 29 (here, a 640×480 pixel CMOS camera sensor) at a distal end of the housing portion. The underside of the blade further comprises a laterally-directed guide face 19 for guiding intubation. This is conveniently formed in the present embodiment as a side wall of the housing portion 17.

The blade is conveniently formed from a cast or moulded zinc alloy, although the material of the blade is not particularly limited. The total vertical extent of the blade portion 7 at any one point measured along the longitudinal length of the blade between the underside and the upper side of the blade is 10 mm or less. This low vertical profile allows for greater freedom of movement during the intubation process given the limited space within which the clinician can work.

The handle portion 5 and the blade portion 7 are fixedly connected at the handle connection end 11 of the blade. The handle portion 5 is generally barrel-shaped, which allows for greater comfort during use. The handle portion 5 comprises a connection section 21 configured to provide a mechanical and electrical connection of the handle portion to the image display module 3. The connection section 21 includes a USB connector 23 arranged for connection with a corresponding USB connector port (not visible) on the image display module 3. Whilst in this arrangement, the USB connector on the handle is a ‘male’ USB connector configured to engage with a ‘female’ USB port on the image display module, it will be understood that an alternative configuration may be used in practice (e.g. provision of a ‘female’ USB port on the handle portion, and provision of a ‘male’ USB connector on the image display module, or provision of an alternative (non-USB) type of electrical connection).

The laryngoscope body 1 contains no internal power source. Rather, the sole power source of the laryngoscopy system is the internal battery of the image display module 3, which is a non-removable, rechargeable battery. All powered components which form part of the laryngoscopy body, for example the light emitting components 28, and the imaging component 29, are therefore powered by the image display module, e.g. via one or more internal wires. This arrangement reduces the weight of the laryngoscope body, and leads to reductions in the cost of manufacture of the laryngoscope body, which makes it suitable for use as a ‘single use’, or disposable component.

The image display module 3 comprises a module housing 25 and a screen 27. The image display module is configured to engage with the handle portion of the laryngoscope body such that a median plane of a screen portion of the image display (as indicated by line M₁) is offset from a median plane of the handle portion (as indicated by line M₂) of the laryngoscope body. This arrangement provides increase ease of manipulation of the laryngoscope in response to the observed video being displayed on the image display module.

The image display module has no external control members, i.e. no buttons, switches, dials formed on or disposed on an outer surface of the module. This allows for reduced contamination risk. The image display module does comprise an internal (recessed) device reset button 26, accessible via a pin-hole opening formed in the outer surface of the image display module. As discussed above, in some arrangements, the image display module may comprise a single external control member, such as an on/off power button, but such an arrangement is not shown in the figures.

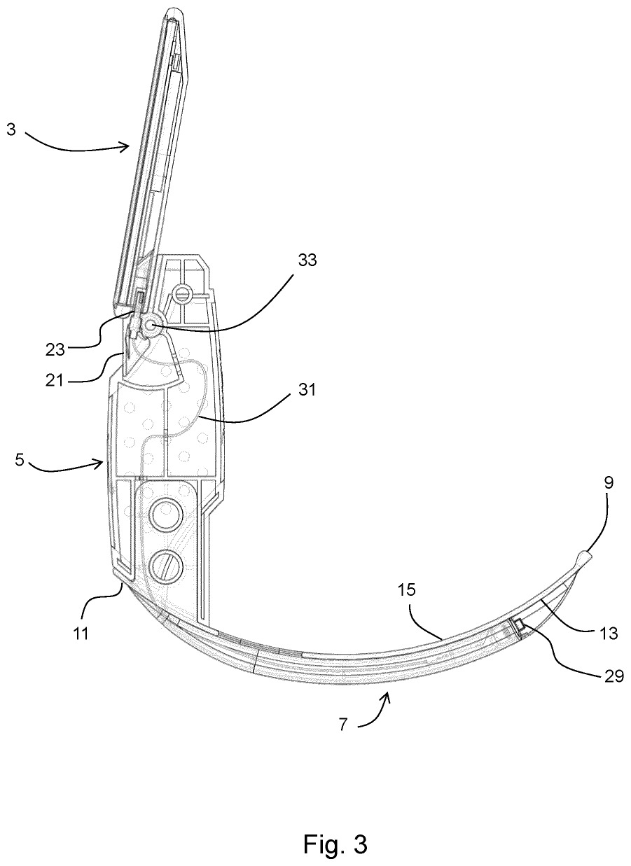

The image display module includes an accelerometer (not shown) for motion input control of the image display module, in a manner discussed below. The image display module is configured to receive image data from the imaging component 29 disposed within the housing portion of the laryngoscope body. Conveniently, the image display module is configured to receive image data via one or more internal wires 31 disposed within the laryngoscope body, as shown in FIG. 3, which is a partial cutaway line drawing of the laryngoscopy system of FIGS. 1 and 2. Said internal wire(s) are connected at a first end to the imaging component 29, and connected at a second end to the USB connector 23. Image data can then be transferred to the image display module via the USB connection. It is also contemplated that in some arrangements, the image display module may be configured to receive image data by wireless transmission from the imaging component.

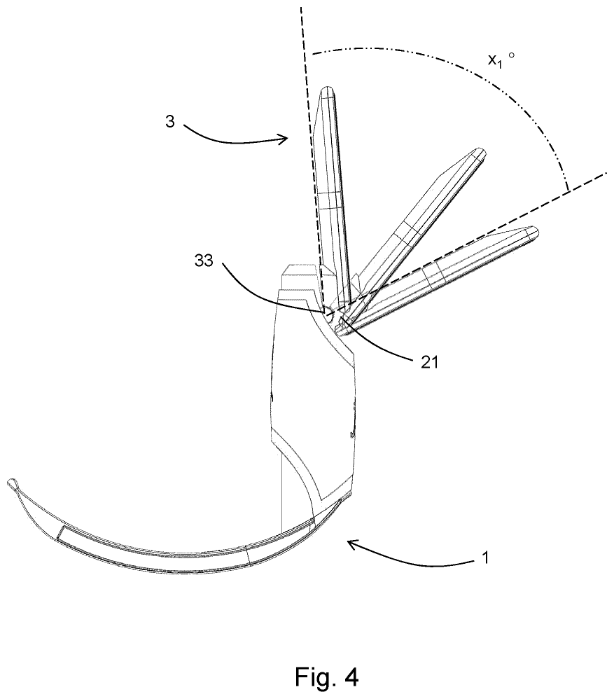

As shown in FIG. 4, the image display module 3 is, in this arrangement, pivotably mounted on the handle portion 5 of the laryngoscope body 1. The pivoting connection is provided by means of a pivot hinge 33 formed on the handle portion of the laryngoscope body, the connection section 21 of the handle portion being pivotable relative to the remainder of the handle portion. As the image display module is mounted to the connection section 21 of the handle portion, this therefore allows for pivoting of the image display module relating to the handle portion through an angle of x₁°. A pivotable mounting provides advantages in terms of ease of use of the system, as a user of the device can adjust the angle of the image display module relative to the laryngoscope body to thereby select a comfortable viewing angle of the image display module during use.

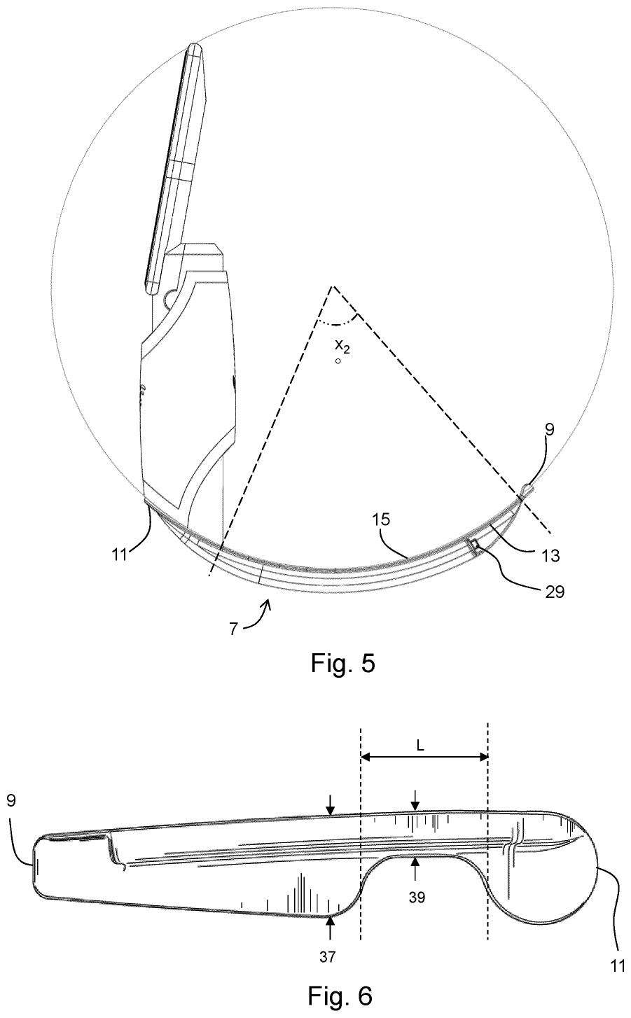

Another aspect of the present invention relates to a laryngoscopy system in which an upper side of a blade body of the laryngoscope defines a generally longitudinally arcuate surface, and at least a portion of the surface has a constant radius of curvature. FIG. 5 shows a side view line drawing of a laryngoscopy system having such an arrangement. The constant radius of curvature is defined based on the upper side 15 of the blade body; that is, the upper side 15 of the blade body defines a generally longitudinally arcuate surface, and a portion of this surface subtending an angle of x₂° has a constant radius of curvature. In this arrangement, x₂ is about 65°, however the constant radius of curvature portion may subtend any angle between 30° up to 90° or more. Providing a blade with such a curvature can allow for ease of insertion of the laryngoscope blade to a patients airway during e.g. an intubation procedure.

Another aspect of the present invention relates to a laryngoscopy system in which the blade body of the laryngoscopy system has a locally narrowed portion disposed adjacent the handle connection end of the blade. FIG. 6 shows a bottom view line drawing of such a blade. The lateral extent of the locally narrowed portion 35 is less than 60% of the maximum lateral extent 37 of the blade body. In the arrangement shown here, the blade is asymmetrically narrowed such that the lateral extent of the locally narrowed portion is about 11 mm, and the maximum lateral extent of the blade body is about 25 mm. In this way, the lateral extent of the locally narrowed portion is about 44% of the maximum lateral extent of the blade body.

The length (L) of the locally narrowed portion is selected to allow for reduced impingement on a patient's teeth during intubation of the patient. This can reduce risk of damage to a patient's teeth during use of the laryngoscope, and furthermore provide greater range of movement for a user during an intubation procedure. The length of the locally narrowed portion is defined between first and second points at which the lateral extent of the blade is 60% or less of the maximum lateral extent, as indicated approximately by the two dotted lines in FIG. 6. In the arrangement shown, the length of the locally narrowed portion is approximately 30 mm.

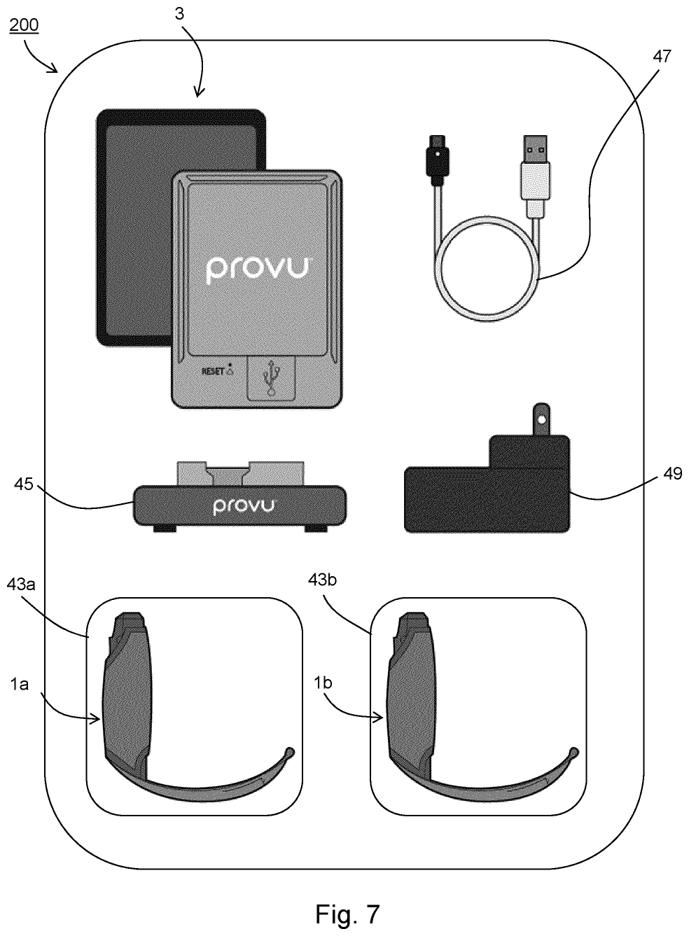

Laryngoscopy systems according to the present invention may be provided in kit form. FIG. 7 shows a schematic drawing of a kit according to the present invention. The kit comprises a single image display module 3 (both sides of the image display module shown in FIG. 7), the image display module having features as described above. The kit further includes two laryngoscope bodies 1 which are provided in respective sealed and sterile containers 43a,b, the laryngoscope bodies having features as discussed above. Providing a kit in this manner means that a plurality of sterile laryngoscope bodies 1a, b can be used as single-use laryngoscope bodies, in combination with the single reusable and sterilisable image display module 3.

In the embodiment shown in FIG. 7, the kit further comprises a charging stand 45, configured to engage with the image display module to charge the internal battery of the image display module, a USB cable 47 and a mains power supply connector 49. The internal battery of the image display module can be charged or recharged by connection to an external power source, e.g. by engaging the image display module with the charging stand, and connecting the charging stand to a mains power supply using the mains power supply connector. It may be necessary to charge the image display module before first use, in particular in cases where the image display module is placed in a ‘hard off’ mode during manufacture. In such a case, the image display module may be configured to be placed in a standard operational mode upon connection to a power source for charging.

To ready the kit for use in a laryngoscopy procedure, an operator (e.g. a clinician) opens at least one of the sealed containers 43a, and extracts the laryngoscope body 1a, in sterile condition. The laryngoscope body is then connected to the image display module 3 by engagement of the USB connector on the laryngoscopy body with the cooperating USB port on the image display module, to provide a laryngoscopy system 100 ready for use in e.g. a tracheal intubation procedure as shown in FIG. 8.

As the image display module is picked up by the clinician, the accelerometer of the image display module detects this motion activity, thereby triggering the image display module to enter the active mode. Upon entering the active mode, the processor of the image display module sets a movement level threshold, and updates a system timer status to a predetermined time value of 60 seconds. When the image display module is in active mode, and connected to the laryngoscopy body, a screen portion of the image display module is configured to receive and display image data transmitted from the imaging component on the screen portion of the image display module for viewing by the clinician. Power is also supplied to the light emitting component (LED) at the distal end of the blade of the laryngoscopy body, to assist in visibility during the procedure.

To perform a tracheal intubation, the clinician (represented by the eye motif in FIG. 8) standing above/behind the head of a supine patient then holds the laryngoscopy system in one hand, blade-downwards with the blade underside 13 away from them, and an endotracheal tube (ET tube) in the other hand (not shown). The blade portion 7 of the laryngoscope body is then inserted into the patient's airway 40, and may be turned and/or lifted to move tissues such as the tongue and epiglottis out of the way, to provide an imaged line of sight along the patient's airway to the patient's vocal cords 41. This allows the clinician to visualise the ET tube as it is passed through the patient's vocal cords into the trachea.

During this procedure, the detected movement activity resulting from movement of the laryngoscopy system by the clinician will generally exceed the movement level threshold of the system, and thus the system will remain in an active mode throughout the procedure. The system timer status is also updated, each time the movement threshold is exceeded. If the system is moved continuously it will therefore remain active. If the system is unmoved, or the detected motion activity does not exceed the movement threshold for a time period of 60 seconds, the image display module is configured to fade the screen of the image display module to black for 5 seconds and then enter standby mode or sleep mode, in which the display of the image display module is turned off, and in which the processor of the image display module is not active, thereby preserving battery life of the system during periods of non-use.

After use, the laryngoscope body 1a will be contaminated. However, rather than subjecting these elements of the system to cleaning and attempting to sterilize them, they are disposed of after a single use. This allows these elements of the system to have a simpler structure than conventional reusable laryngoscope bodies, since they need not withstand repeated cleaning operations. The (reusable) image display module 3 can then be cleaned and/or sterilized for future use in a conventional manner.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

1. A laryngoscopy system comprising:
a laryngoscope body having a handle portion and a blade portion, the blade portion having a distal end and a handle connection end and a blade body extending therebetween, the laryngoscope body further comprising an imaging component disposed on or housed by the blade portion; and
an image display module configured to receive image data from the imaging component;
wherein the image display module includes an accelerometer for motion input control of the image display module.

2. A laryngoscopy system according to claim 1 wherein the operational status of the image display module is controllable based on motion detection by the accelerometer.

3. A laryngoscopy system according to claim 1 wherein the image display module and/or the laryngoscope body has no external buttons, switches or dials.

4. A laryngoscopy system according to claim 1 wherein the image display module comprises an integral power source, optionally wherein the integral power source is non-removable.

5. A laryngoscopy system according to claim 1 wherein the integral power source of the image display module is the sole power source for the laryngoscopy system.

6. A laryngoscopy system according to claim 1 wherein the image display module has an ingress protection rating as defined in international standard IEC 60529 of IP64 or higher.

7. A laryngoscopy system according to claim 1 wherein the image display module is pivotably mounted on the handle portion of the laryngoscope body, optionally through a pivot hinge.

8. A laryngoscopy system according to claim 1 wherein the image display module is removably mountable on the handle portion of the laryngoscope body.

9. A laryngoscopy system according to claim 1 wherein the image display module is mounted to the handle by corresponding male and female electrical connectors, optionally wherein the electrical connectors are corresponding male and female USB connectors.

10. A laryngoscopy system according to claim 1 wherein the laryngoscope body comprises a light emitting component disposed on or housed by the blade portion.

11. A laryngoscopy system according to claim 1 wherein the field of view of the imaging component includes part of the blade portion of the laryngoscope body.

12. A laryngoscopy system according to claim 1 wherein the imaging component is a camera disposed within a housing section of the blade portion of the laryngoscope body.

13. A laryngoscopy system according to claim 1 wherein the blade body has a locally narrowed portion disposed adjacent the handle connection end of the blade, the lateral extent of the locally narrowed portion being less than 60% of the maximum lateral extent of the blade body.

14. A laryngoscopy system according to claim 13 wherein the locally narrowed portion is formed asymmetrically with respect to the longitudinal axis of the blade body.

15. A laryngoscopy system according to claim 13 wherein the length of the locally narrowed portion is in a range from 1 cm to 5 cm in length.

16. A laryngoscopy system according to claim 1 wherein an upper side of the blade body defines a generally longitudinally arcuate surface, and at least a portion of the surface subtending an angle of at least 30° has a constant radius of curvature.

17. A laryngoscopy system according to claim 16 wherein the portion of constant radius of curvature is located towards the distal end of the blade.

18. (canceled)

19. A laryngoscopy system according to claim 1 wherein the image display module is configured to engage with the handle portion of the laryngoscope body such that a median plane of a screen portion of the image display intersects with a longitudinal central portion of the blade body of the laryngoscope.

20. An image display module configured for engagement with a laryngoscope body comprising an imaging component to thereby form a laryngoscopy system;
wherein the image display module is configured to receive image data from the imaging component of the laryngoscope body when engaged with the laryngoscope body; and
wherein the image display module comprises an accelerometer for motion input control of the image display module screen.

21. A kit comprising an image display module according to claim 20 and at least one laryngoscope body, the laryngoscope body having a handle portion and a blade portion, the blade portion having a distal end and a handle connection end and a blade body extending therebetween, the laryngoscope body further comprising an imaging component and a light emitting component disposed on or housed by the blade portion.

22. (canceled)
